(19) United States
(12) Patent Application Publication
(10) Pub. No.: US 2026/0310825 A1
(43) Pub. Date: Oct. 8, 2026

(54) SYSTEM AND METHOD FOR PRIVACY-PRESERVING SOCIAL ACCOUNTABILITY IN BEHAVIORAL HABIT FORMATION

(71) Applicant: Savrr Inc., Spring, TX (US)

(72) Inventors: Jamilah Jafun, Spring, TX (US); Oluwanisola Ola, Spring, TX (US)

(21) Appl. No.: 19/457,564

(22) Filed: Jan. 23, 2026

Related U.S. Application Data
(60) Provisional application No. 63/749,099, filed on Jan. 24, 2025.

(51) Int. Cl. G06F 21/62

(52) U.S. Cl. CPC G06F 21/6254

(57) ABSTRACT

A computer-implemented system for behavioral habit formation with privacy-preserving social accountability comprises a processor and memory storing instructions that, when executed, cause the system to receive data defining a goal associated with a first user. The instructions cause the system to determine goal-related data based on user-provided data or data from user-linked resources. The instructions maintain a circle data structure identifying invited participants and receive a visibility designation indicating whether the goal is shared. When the goal is shared, the system transmits to participant devices an abstracted progress signal derived from the goal, comprising abstracted progress information while excluding sensitive goal data.

### CROSS-REFERENCE TO RELATED APPLICATIONS

This application claims priority to U.S. Provisional Application No. 63/749,099, titled Peer Community Financial Management and Accountability, filed Jan. 24, 2025, which is hereby incorporated by reference in its entirety.

### FIELD OF INVENTION

The present disclosure relates to computer-implemented systems and methods for behavioral habit formation, and more particularly to a privacy-preserving social accountability platform that enables users to share abstracted progress signals with invited participants without exposing sensitive underlying data.

### BACKGROUND

Behavioral habit formation, particularly in the context of personal finance, presents challenges for individuals seeking to establish and maintain consistent practices over time. Many people struggle to develop sustainable habits related to saving, budgeting, and financial planning without external support structures or accountability mechanisms.

Social accountability has long been recognized as a motivating factor in behavior change. When individuals share their goals or progress with others, they may experience increased motivation to follow through on commitments. Traditional approaches to social accountability in financial contexts, however, often involve disclosure of sensitive financial information, including account balances, transaction histories, and specific monetary amounts. Such disclosure can create privacy concerns and may discourage individuals from seeking social support for their financial goals.

Existing systems for goal tracking and social motivation typically fall into several categories. Some systems operate in isolation, providing users with tools to set and track goals without any social component. Other systems enable full sharing of goal details and progress with social connections, but do so without meaningful privacy protections. Group-based financial tools, such as pooled savings accounts or collective investment platforms, involve commingling of funds and shared access to financial resources, which may not align with the preferences of users who wish to maintain individual control over their finances while still benefiting from social motivation.

The intersection of behavioral science and technology has produced various approaches to encouraging positive habits. These approaches may include reminder notifications, progress tracking, gamification elements such as badges or rewards, and educational content delivery. However, integrating social accountability into such systems while maintaining user privacy presents technical and architectural challenges. Conventional system designs may not adequately separate sensitive underlying data from the progress information that could be shared with others for motivational purposes.

Additionally, users often have varying preferences regarding which goals they wish to share and with whom. A user may desire to share progress on one goal with certain individuals while keeping other goals private or sharing them with different groups. Existing systems may lack the flexibility to accommodate such granular control over visibility settings on a per-goal basis.

There remains room for improvement in systems and methods that combine behavioral habit formation techniques with social accountability while preserving user privacy and providing flexible control over information sharing.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

According to an aspect of the present disclosure, a computer-implemented system for behavioral habit formation with privacy-preserving social accountability is provided. The system comprises a processor and a memory coupled to the processor and storing instructions that, when executed by the processor, cause the system to receive data defining a goal associated with a first user. The instructions further cause the system to determine goal-related data based on at least one of user-provided data or data associated with one or more user-linked resources. The instructions further cause the system to maintain a circle data structure associated with the first user, the circle data structure identifying one or more invited participants. The instructions further cause the system to receive a visibility designation indicating whether the goal is shared with the one or more invited participants. In response to the visibility designation indicating the goal is shared, the instructions cause the system to transmit to devices associated with the one or more invited participants an abstracted progress signal derived from the goal, wherein the progress signal comprises abstracted progress information and excludes sensitive data associated with the goals.

According to other aspects of the present disclosure, the system may include one or more of the following features. The progress signal may comprise at least one of a milestone indicator, a percentage toward goal completion, or a streak indicator. The instructions may further cause the system to generate the progress signal by applying a data abstraction layer that transforms the progress data into abstracted progress information. The circle data structure may be independent from circle data structures associated with other users, such that each user maintains a separate and non-pooled social graph. The instructions may further cause the system to receive data defining a plurality of goals associated with the first user, receive a respective visibility designation for each goal of the plurality of goals, and enforce goal-level visibility rules such that different goals are shared with different subsets of the one or more invited participants. Each goal of the plurality of goals may be associated with a goal state selected from a group comprising created, active, paused, completed, and abandoned. The instructions may further cause the system to detect at least one of user inactivity or goal stagnation and, in response to detecting the at least one of user inactivity or goal stagnation, generate and transmit a behavioral nudge to a user device associated with the first user. The instructions may further cause the system to award a digital badge to the first user based on a defined behavior, the defined behavior comprising at least one of consistency in savings activity, maintenance of a streak, or goal completion. The instructions may further cause the system to generate a total goal representation comprising a first goal portion allocated toward the goal and a second goal portion not allocated toward the goal, wherein the total goal representation is informational and does not modify underlying user data or access to external resources. The instructions may further cause the system to receive allocation data indicating at least one of a manual allocation or an automatic allocation of progress toward the goal and update the first goal portion based on the allocation data without modifying access to the circle data structure.

According to another aspect of the present disclosure, a computer-implemented method for providing social accountability in habit formation while preserving privacy is provided. The method comprises receiving, by a computing system, goal data defining a goal associated with a first user. The method further comprises calculating, by the computing system, goal progress based on data linked to the first user. The method further comprises storing, by the computing system, a circle data structure associated with the first user, the circle data structure identifying a plurality of invited participants. The method further comprises receiving, by the computing system, a sharing designation indicating that the goal is to be shared with the plurality of invited participants. The method further comprises transmitting, by the computing system, the goal progress data to devices associated with the plurality of invited participants, wherein the transmitting excludes transmission of sensitive underlying data associated with the goal.

According to other aspects of the present disclosure, the method may include one or more of the following features. The circle data structure may be independent from circle data structures associated with other users, such that each user maintains a separate and non-pooled social graph for social accountability, the circle data structure comprises unidirectional visibility, the circle data structure comprises viewer-specific views, or the circle data structure comprises asymmetric disclosure. The goal progress may comprise abstracted progress output and the abstracted progress output may comprise at least one of a milestone indicator, a percentage value, or a streak indicator. The method may further comprise generating, by the computing system, a total goal representation comprising a first goal portion allocated toward the goal and a second goal portion not allocated toward the goal, wherein the total goal representation is informational and does not modify access to the circle data structure. The method may further comprise detecting, by the computing system, at least one of user inactivity or goal stagnation based on the goal progress and, in response to detecting the at least one of user inactivity or goal stagnation, generating and transmitting, by the computing system, a behavioral nudge to a device associated with the first user. The method may further comprise awarding, by the computing system, a digital badge to the first user based on a defined behavior, the defined behavior comprising at least one of consistency in activity, maintenance of a goal streak, or goal completion.

According to another aspect of the present disclosure, a non-transitory computer-readable medium storing instructions that, when executed by one or more processors, cause the one or more processors to perform operations is provided. The operations comprise maintaining a goal for a user. The operations further comprise maintaining a user-specific circle data structure identifying invited participants. The operations further comprise storing a visibility setting indicating whether the goal is shared with participants identified in the user-specific circle data structure. The operations further comprise generating, for a shared goal, an abstracted progress signal based on user-specific data. The operations further comprise transmitting the abstracted progress signal to devices associated with the invited participants.

According to other aspects of the present disclosure, the non-transitory computer-readable medium may include one or more of the following features. The abstracted progress signal may comprise at least one of a milestone indicator, a percentage toward goal completion, or a streak indicator. The circle data structure may be independent from circle data structures associated with other users, such that each user maintains a separate and non-pooled social graph. The operations may further comprise detecting at least one of user inactivity or goal stagnation and, in response to detecting the at least one of user inactivity or goal stagnation, generating and transmitting a behavioral nudge to a device associated with the user.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 illustrates a system environment for privacy-preserving social accountability in behavioral habit formation, according to aspects of the present disclosure.

FIG. 2 illustrates a block diagram of an application server and associated components, according to aspects of the present disclosure.

FIG. 3 illustrates a flowchart for a method for generating and transmitting abstracted progress signals, according to aspects of the present disclosure.

FIG. 4 illustrates a flowchart for a method for generating and delivering behavioral feedback, according to aspects of the present disclosure.

FIG. 5 illustrates a block diagram of a system for providing notifications and messaging, according to aspects of the present disclosure.

FIG. 6 illustrates a flowchart for a method for behavioral feedback in a habit formation system, according to aspects of the present disclosure.

FIG. 7 illustrates a block diagram of a system for managing user circles and privacy-preserving social accountability, according to aspects of the present disclosure.

FIG. 8 illustrates a flowchart for a method for providing social accountability while preserving privacy, according to aspects of the present disclosure.

FIG. 9 illustrates a flowchart for a method for behavioral habit formation with privacy-preserving social accountability, according to aspects of the present disclosure.

FIG. 10 illustrates a flowchart for a method for providing social accountability in habit formation, according to aspects of the present disclosure.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure relates to systems and methods for behavioral habit formation with privacy-preserving social accountability. In various implementations, a computing system may enable users to establish goals and share progress toward those goals with invited participants while preserving the privacy of sensitive underlying data. The system may transform sensitive data associated with user goals into abstracted progress signals that convey progress information without revealing the sensitive data from which the progress signals are derived.

In some cases, the system may maintain a user-specific circle data structure for each user. The user-specific circle data structure may identify one or more invited participants with whom the user may share goal progress. Each user may maintain a separate circle data structure that is independent from circle data structures associated with other users. In this manner, each user may maintain a non-pooled social graph for social accountability purposes. The circle data structure may support unidirectional visibility, asymmetric disclosure, or viewer-specific views, such that a first user may expose progress for one or more goals to members of a selected circle without receiving access to goals of those members unless such members independently designate goals as shared with the first user.

The system may receive visibility designations indicating whether particular goals are to be shared with invited participants identified in the user-specific circle data structure. In some implementations, the system may support goal-level visibility rules such that different goals associated with a single user may be shared with different subsets of invited participants. A user may designate some goals as private and other goals as shared, and the system may enforce these visibility designations when determining whether to transmit progress information to invited participants.

When a goal is designated as shared, the system may generate an abstracted progress signal derived from goal-related data. The abstracted progress signal may comprise abstracted progress information such as a milestone indicator, a percentage toward goal completion, or a streak indicator. The abstracted progress signal may exclude sensitive data associated with the goal, such as any behavioral goal indicated by the user, or, in a financial context, account balances, transaction histories, or other financial information. In this manner, the system may enable social accountability through visibility of progress rather than through disclosure of sensitive underlying data. In some implementations, visibility of abstracted progress information may be delayed, conditional, or event-triggered, such that progress information is disclosed only after predefined conditions are met. In some implementations, the abstracted progress information may be generated using any transformation, mapping, categorization, or summarization of underlying goal data, including symbolic, qualitative, probabilistic, or state-based representations, such that the abstracted progress information conveys progress without revealing underlying sensitive data. The specific form of abstraction may vary across implementations and is not limited to any particular representation.

The system may determine goal-related data based on user-provided data or data associated with one or more user-linked resources. In some cases, the system may retrieve data from external data sources to determine goal progress. The system may apply a data abstraction layer that transforms the goal-related data into abstracted progress information suitable for transmission to invited participants. The data abstraction layer may enforce privacy rules that govern what information is included in the abstracted progress signal and what information is excluded.

In various implementations, the system may support behavioral reinforcement mechanisms to encourage continued progress toward goals. The system may detect user inactivity or goal stagnation and, in response, generate and transmit a behavioral nudge to a device associated with the user. The system may also award digital badges to users based on defined behaviors such as consistency in activity, maintenance of a goal streak, or goal completion. These behavioral reinforcement mechanisms may operate in conjunction with the social accountability features to support behavioral habit formation.

The system may maintain a total goal representation comprising a first goal portion allocated toward a goal and a second goal portion not allocated toward the goal. The total goal representation may be informational and may not modify underlying user data or access to external resources. In some cases, the system may receive allocation data indicating a manual allocation or an automatic allocation of progress toward the goal and may update the first goal portion based on the allocation data. The allocation functionality may enable users to mentally partition available resources toward goals without requiring custodial control over those resources.

While various examples described herein relate to financial habits such as saving, budgeting, and financial planning, the systems and methods for privacy-preserving social accountability are not limited to financial contexts. For example, the goal may relate to educational progress, health routines, or professional development, with abstracted progress information reflecting completion states or consistency indicators rather than numerical measurements. The circle management functionality and associated privacy-preserving mechanisms may be applied to a wide variety of behavioral habits and goal types. In some aspects, the system may support health and wellness goals such as exercise routines, dietary habits, sleep patterns, or meditation practices. In some cases, the system may facilitate educational goals such as study habits, skill development, or learning milestones. The system may also support productivity-related goals, personal development objectives, or any other behavioral habits where social accountability may provide motivational benefits. The financial habit examples described herein serve as illustrative implementations of the broader privacy-preserving social accountability framework, and the abstracted progress signals, circle data structures, visibility designations, and behavioral reinforcement mechanisms may be adapted to support habit formation across diverse domains without departing from the principles described in this disclosure.

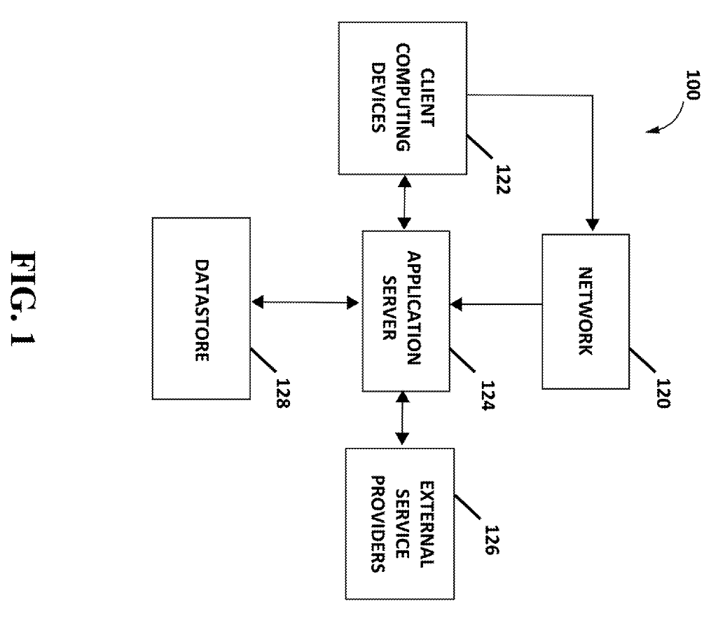

Referring to FIG. 1, a system environment 100 for privacy-preserving social accountability in behavioral habit formation is illustrated. The system environment 100 may comprise a computer-implemented system for behavioral habit formation with privacy-preserving social accountability. The system environment 100 may include a network 120, client computing devices 122, an application server 124, external service providers 126, and a datastore 128. The various components of the system environment 100 may communicate with one another through the network 120.

The network 120 may serve as a communication infrastructure connecting the various components of the system environment 100. The network 120 may comprise one or more wired or wireless networks, including local area networks, wide area networks, cellular networks, the Internet, or combinations thereof. The network 120 may facilitate data transmission between the client computing devices 122, the application server 124, the external service providers 126, and the datastore 128.

With continued reference to FIG. 1, the client computing devices 122 may be connected to the network 120 and may include devices such as smartphones, tablets, or personal computers through which users interact with the system environment 100. The client computing devices 122 may execute client applications that provide user interfaces for establishing goals, managing circle data structures, setting visibility designations, and viewing abstracted progress signals. Each client computing device of the client computing devices 122 may include a processor and a memory coupled to the processor. The memory may store instructions that, when executed by the processor, cause the client computing device to perform operations associated with behavioral habit formation and social accountability.

The application server 124 may be connected to the network 120 and may communicate with the client computing devices 122, the external service providers 126, and the datastore 128. The application server 124 may serve as a central component that processes requests from the client computing devices 122. The application server 124 may include a processor and a memory coupled to the processor. The memory may store instructions that, when executed by the processor, cause the application server 124 to manage goal data, circle data structures, and visibility settings. The application server 124 may generate abstracted progress signals for transmission to invited participants based on goal-related data and visibility designations. As further shown in FIG. 1, the application server 124 may coordinate data flows between the client computing devices 122, the external service providers 126, and the datastore 128.

The application server 124 may be implemented using various hardware, firmware, and software components. In some aspects, the application server 124 may comprise one or more physical servers, virtual machines, or cloud-based computing instances. The processor of the application server 124 may include one or more central processing units (CPUs), graphics processing units (GPUs), application-specific integrated circuits (ASICs), field-programmable gate arrays (FPGAs), or other processing elements capable of executing instructions. The processor may be a single-core or multi-core processor and may operate at various clock speeds depending on the computational requirements of the system.

The memory coupled to the processor may include volatile memory such as random access memory (RAM), dynamic random access memory (DRAM), or static random access memory (SRAM). The memory may also include non-volatile memory such as read-only memory (ROM), electrically erasable programmable read-only memory (EEPROM), flash memory, or other persistent storage media. In some implementations, the memory may comprise a combination of volatile and non-volatile memory components arranged in a memory hierarchy that includes cache memory, main memory, and secondary storage.

The application server 124 may include firmware stored in non-volatile memory that provides low-level control for the hardware components. The firmware may include a basic input/output system (BIOS) or unified extensible firmware interface (UEFI) that initializes hardware components during system startup. The firmware may also include embedded software that controls specific hardware functions or provides hardware abstraction layers for higher-level software components.

In some cases, the application server 124 may include one or more network interface controllers (NICs) that enable communication over the network 120. The network interface controllers may support wired connections such as Ethernet or fiber optic connections, or wireless connections such as Wi-Fi or cellular connectivity. The application server 124 may also include storage controllers that manage access to the datastore 128 or local storage devices.

The application server 124 may include an operating system that manages hardware resources and provides services for application software. The operating system may be a general-purpose operating system such as Linux, Windows Server, or a Unix-based system, or may be a specialized operating system designed for server environments. The instructions stored in the memory may include application software that executes on the operating system to perform the operations described herein, including receiving goal data, maintaining circle data structures, processing visibility designations, and generating abstracted progress signals.

The client computing devices 122 may similarly include processors, memory, firmware, and other hardware components. The processors of the client computing devices 122 may include mobile processors, system-on-chip (SoC) devices, or desktop processors depending on the form factor of the device. The memory of the client computing devices 122 may include RAM for active processing and flash storage or solid-state drives for persistent data storage. The client computing devices 122 may include display screens, input devices such as touchscreens or keyboards, and wireless communication modules for connecting to the network 120.

The external service providers 126 may be connected to the network 120 and to the application server 124. The external service providers 126 may provide services such as financial data retrieval or notification delivery. In some implementations, the external service providers 126 may integrate with third-party financial data providers using vendor-agnostic functional integration patterns. The vendor-agnostic functional integration patterns may enable the application server 124 to retrieve data from various external sources without being dependent on specific vendors or geographic regions. The external service providers 126 may supply user-linked resource data that the application server 124 may use to determine goal-related data and calculate goal progress.

The datastore 128 may be connected to the application server 124 and may store user data, goal data, circle data structures, visibility designations, and other information used by the system environment 100. The datastore 128 may comprise one or more databases, file systems, or other persistent storage mechanisms. The application server 124 may retrieve data from the datastore 128 when processing requests from the client computing devices 122 and may store updated data to the datastore 128 as users establish goals, modify visibility settings, or update circle data structures.

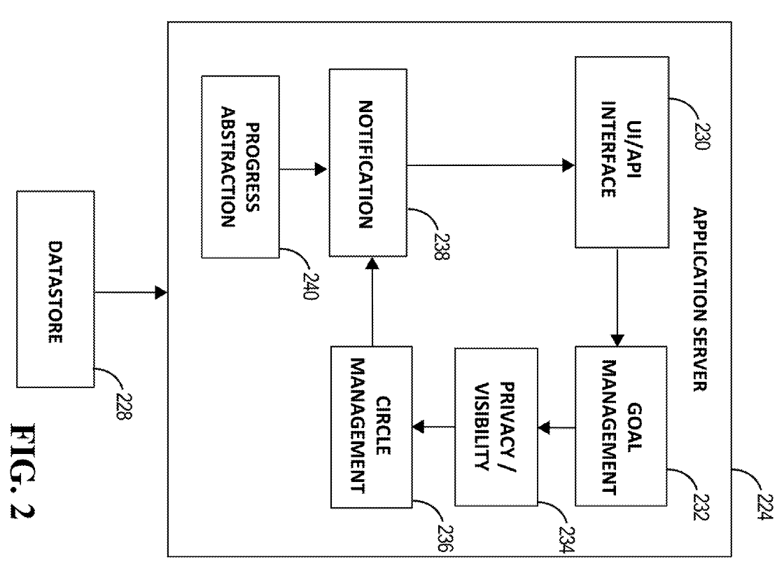

Referring to FIG. 2, a block diagram of an application server 224 and associated components for a privacy-preserving social accountability system is illustrated. The application server 224 may comprise a computer-implemented system for behavioral habit formation with privacy-preserving social accountability. The application server 224 may include several interconnected modules that work together to manage goals, privacy settings, social circles, notifications, and progress abstraction. The application server 224 may be connected to a datastore 228 for persistent data storage. The datastore 228 may store user data, goal data, circle data structures, visibility settings, progress information, and other system data used by the various modules within the application server 224.

With continued reference to FIG. 2, the application server 224 may comprise a UI/API interface 230, a goal management module 232, a privacy/visibility module 234, a circle management module 236, a notification module 238, and a progress abstraction module 240. The UI/API interface 230 may serve as an entry point for user interactions and external communications with the system. The UI/API interface 230 may receive requests from client computing devices and may provide responses containing goal data, progress information, and other data requested by users. The UI/API interface 230 may also facilitate communication with external service providers for retrieving data associated with user-linked resources.

As used herein, the term "module" may refer to, be part of, or include an application-specific integrated circuit (ASIC), an electronic circuit, a combinational logic circuit, a field programmable gate array (FPGA), a processor (shared, dedicated, or group) that executes code, other suitable hardware components that provide the described functionality, or a combination of some or all of the above, such as in a system-on-chip. A module may include memory (shared, dedicated, or group) that stores code executed by the processor. The term "code" may include software, firmware, bytecode, or a combination thereof. The term "processor" may include a microprocessor, a microcontroller, a digital signal processor (DSP), or any other programmable device capable of executing instructions.

The goal management module 232 may be connected to the UI/API interface 230 and may handle creation, modification, and tracking of user goals. The goal management module 232 may receive data defining a goal associated with a first user through the UI/API interface 230. The goal management module 232 may support multiple concurrent goals per user, allowing users to manage several savings objectives simultaneously within the system. In some implementations, the goal management module 232 may receive data defining a plurality of goals associated with the first user and may maintain each goal independently within the datastore 228.

The goal management module 232 may support both time-bound goals with defined deadlines and open-ended goals without specific completion dates. Time-bound goals may include a target date by which the user intends to achieve the goal, while open-ended goals may allow users to progress toward objectives without temporal constraints. The goal management module 232 may implement a goal state machine with defined states including created, active, paused, completed, and abandoned. Each goal of the plurality of goals may be associated with a goal state selected from a group comprising created, active, paused, completed, and abandoned. The goal state machine may enable dynamic goal lifecycle management by tracking transitions between states as users interact with their goals.

As further shown in FIG. 2, the privacy/visibility module 234 may be connected to the goal management module 232 and may manage visibility settings and privacy rules associated with each goal. The privacy/visibility module 234 may determine what information can be shared and with whom based on user-defined preferences. The application server 224 may employ a separation of financial data, goal data, and social visibility data as distinct data layers within a privacy-by-design architecture. The privacy/visibility module 234 may enforce this separation by maintaining financial data, goal data, and social visibility data in separate logical or physical storage structures within the datastore 228.

The privacy/visibility module 234 may receive a respective visibility designation for each goal of the plurality of goals associated with a user. The privacy/visibility module 234 may enforce goal-level visibility rules such that different goals are shared with different subsets of the one or more invited participants. In this manner, a user may designate some goals as private and other goals as shared with particular invited participants, and the privacy/visibility module 234 may enforce these designations when determining whether to transmit progress information.

With continued reference to FIG. 2, the circle management module 236 may be connected to the privacy/visibility module 234 and may maintain user-specific circle data structures identifying invited participants. The circle management module 236 may maintain a circle data structure associated with the first user, the circle data structure identifying one or more invited participants. In some embodiments, the system operates for a single user without any invited participants, with the social accountability features becoming active only upon user selection. Further, the circle management module 236 may manage the social graph for each user independently, enabling asymmetric or unidirectional social visibility. The circle management module 236 may enable a first user to expose progress for one or more shared goals to circle members without receiving access to any goals of those members unless such members independently designate goals as shared. Unlike systems that rank or compare users relative to one another, the disclosed system may provide accountability signals without computing or displaying comparative rankings among participants. This asymmetric goal sharing functionality may allow users to share their progress without creating reciprocal obligations for invited participants to share their own goals.

The notification module 238 may be connected to the circle management module 236 and to the UI/API interface 230. The notification module 238 may handle generation and transmission of notifications to users and their circle participants. The notification module 238 may deliver notifications through the UI/API interface 230 to client computing devices associated with users and invited participants. The notification module 238 may transmit abstracted progress signals to invited participants when goals are designated as shared.

As further shown in FIG. 2, the progress abstraction module 240 may be connected to the notification module 238 and to the datastore 228. The progress abstraction module 240 may transform sensitive data into abstracted progress signals such as milestone indicators, percentages, or streak indicators. The progress abstraction module 240 may determine goal-related data based on at least one of user-provided data or data associated with one or more user-linked resources. The abstracted progress signals generated by the progress abstraction module 240 may be shared with circle participants through the notification module 238 without exposing underlying sensitive information.

The progress abstraction module 240 may maintain a conceptual bifurcation of user funds into a first balance representation representing funds allocated toward savings goals and a second balance representation representing unallocated funds. This dual balance representation may be informational and may not modify underlying user data or access to external resources. The progress abstraction module 240 may retrieve data from the datastore 228 to calculate progress toward goals and may generate abstracted progress outputs that convey progress information while excluding sensitive financial data such as account balances or transaction histories.

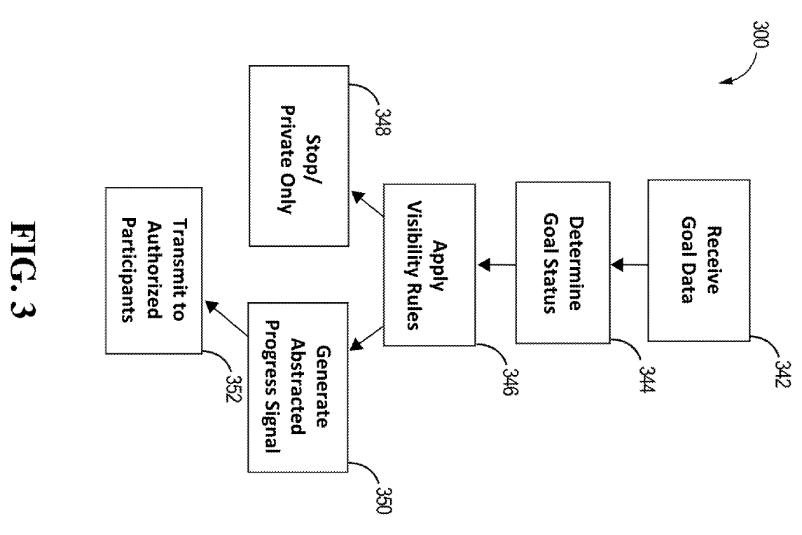

Referring to FIG. 3, a method 300 for generating and transmitting abstracted progress signals based on goal data is illustrated. The method 300 may be performed by a computer-implemented system for behavioral habit formation with privacy-preserving social accountability. In some implementations, the method 300 may be performed by the application server 124 of FIG. 1 or the application server 224 of FIG. 2. The method 300 may enable social accountability through transmission of abstracted progress information while excluding sensitive data associated with goals.

The method 300 may begin with a step 342, where the system receives goal data. The goal data may define a goal associated with a first user. In some cases, the goal data may be received through the UI/API interface 230 from a client computing device associated with the first user. The goal data may include information such as a goal name, a target amount, a target date, or other parameters that define the goal. The goal management module 232 may process the received goal data and store the goal data in the datastore 228.

With continued reference to FIG. 3, the method 300 may proceed to a step 344, where the system determines goal status based on the received goal data. The goal status may indicate the current state of the goal within a goal lifecycle. In some implementations, the goal status may be selected from a group comprising created, active, paused, completed, and abandoned. The goal management module 232 may determine the goal status by evaluating the received goal data against stored goal parameters and progress information retrieved from the datastore 228. The goal status determination may also incorporate data associated with one or more user-linked resources to assess progress toward the goal.

The method 300 may then proceed to a step 346, where the system applies visibility rules to determine how the goal information should be handled. The privacy/visibility module 234 may apply rule-based governance that determines what information is shown, to whom the information is shown, and when the information is displayed. The system may receive a visibility designation indicating whether the goal is shared with one or more invited participants. The visibility rules may be stored in the datastore 228 and may be associated with the goal or with a circle data structure maintained for the first user. The circle data structure may be independent from circle data structures associated with other users, such that each user maintains a separate and non-pooled social graph.

As further shown in FIG. 3, based on the application of visibility rules at the step 346, the method 300 may branch into two paths. In one path, the method 300 may proceed to a step 348, where the process stops or the goal remains private. The step 348 may be reached when the visibility designation indicates that the goal is not shared with invited participants. When the goal remains private, no progress information is transmitted to external participants, and the goal data remains accessible to the goal owner through the client computing devices 122.

In the alternative path, when the visibility designation indicates the goal is shared, the method 300 may proceed to a step 350, where the system generates an abstracted progress signal derived from the goal data. The progress abstraction module 240 may generate the abstracted progress signal by applying a data abstraction layer that transforms sensitive goal-related information into non-sensitive progress indicators. The abstracted progress signal may comprise abstracted progress information such as a milestone indicator, a percentage toward goal completion, or a streak indicator. The data abstraction layer may transform progress data into abstracted progress information that conveys goal progress without revealing sensitive underlying data such as account balances, transaction histories, or other financial information.

Following the generation of the abstracted progress signal at the step 350, the method 300 may continue to a step 352, where the system transmits the abstracted progress signal to authorized participants. The notification module 238 may transmit the abstracted progress signal to devices associated with the one or more invited participants identified in the circle data structure. The transmitted progress signal may comprise abstracted progress information and may exclude sensitive data associated with the goals. In this manner, the method 300 may enable social accountability through visibility of progress rather than through disclosure of sensitive underlying data. The circle management module 236 may identify the authorized participants based on the circle data structure associated with the first user, and the notification module 238 may deliver the abstracted progress signal through the network 120 to client computing devices associated with the authorized participants.

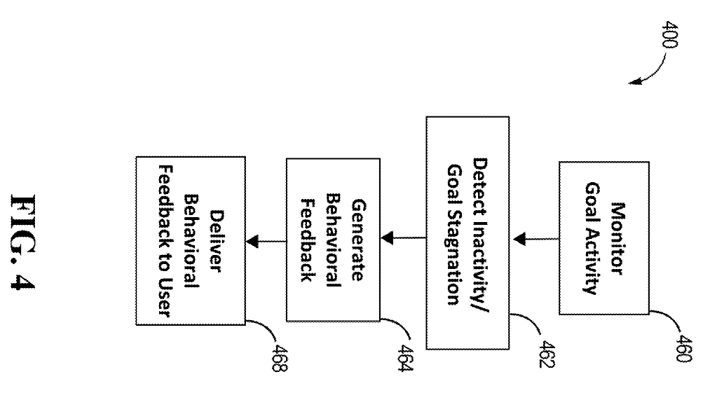

Referring to FIG. 4, a method 400 for generating and delivering behavioral feedback in response to detected inactivity or goal stagnation is illustrated. The method 400 may be performed by a computer-implemented system for behavioral habit formation with privacy-preserving social accountability. In some implementations, the method 400 may be performed by the application server 124 of FIG. 1 or the application server 224 of FIG. 2. The method 400 may represent an adaptive motivation logic that monitors user behavior and goal state to provide timely interventions when progress has stalled, thereby supporting behavioral habit formation through automated reinforcement mechanisms.

The method 400 may begin with a step 460, where the system monitors goal activity associated with a user. The notification module 238 may monitor goal activity by tracking user interactions with goals, progress updates, and other goal-related events stored in the datastore 228. The goal management module 232 may provide goal state information to the notification module 238 to enable monitoring of goal activity over time. In some cases, the monitoring may include tracking the frequency of user interactions with the system, the timing of progress updates, and changes in goal state. The notification module 238 may implement progress-based reinforcement mechanisms that are triggered by user behavior and goal state changes.

With continued reference to FIG. 4, the method 400 may proceed to a step 462, where the system detects inactivity or goal stagnation based on the monitored goal activity. The system may detect at least one of user inactivity or goal stagnation by comparing monitored goal activity against predefined thresholds or patterns. User inactivity may be detected when a user has not interacted with the system or updated goal progress within a specified time period. Goal stagnation may be detected when goal progress has remained unchanged or has decreased over a defined interval despite the goal remaining in an active state. The notification module 238 may evaluate the monitored goal activity data retrieved from the datastore 228 to identify conditions indicative of inactivity or stagnation.

As further shown in FIG. 4, the method 400 may move to a step 464, where the system generates behavioral feedback. In response to detecting the at least one of user inactivity or goal stagnation, the system may generate and transmit a behavioral nudge to a user device associated with the first user. The behavioral feedback may comprise nudges or other reinforcement mechanisms designed to encourage the user to resume progress toward the goal. The notification module 238 may generate the behavioral feedback using progress-based reinforcement triggers that are activated based on the detected inactivity or stagnation conditions. The behavioral feedback may include motivational messages, reminders about goal deadlines, suggestions for actions to advance toward the goal, or other content designed to re-engage the user with the goal.

Following generation of the behavioral feedback at the step 464, the method 400 may proceed to a step 468, where the system delivers the behavioral feedback to the user. The delivery may occur through notifications or other communication channels to a device associated with the user. The notification module 238 may transmit the behavioral nudge through the network 120 to client computing devices 122 associated with the user. The delivery may utilize push notifications, in-application messages, email communications, or other notification mechanisms supported by the system environment 100. The UI/API interface 230 may facilitate delivery of the behavioral feedback to the user device through the appropriate communication channel.

In some implementations, the system may award a digital badge to the first user based on a defined behavior. The defined behavior may comprise at least one of consistency in savings activity, maintenance of a streak, or goal completion. The notification module 238 may evaluate user behavior against defined criteria to determine when a digital badge should be awarded. The digital badges may function as behavioral reinforcement mechanisms that recognize and reward positive behaviors associated with goal progress. The awarded digital badges may be stored in the datastore 228 in association with the user profile and may be displayed through the client computing devices 122 to provide ongoing motivation for continued engagement with goals.

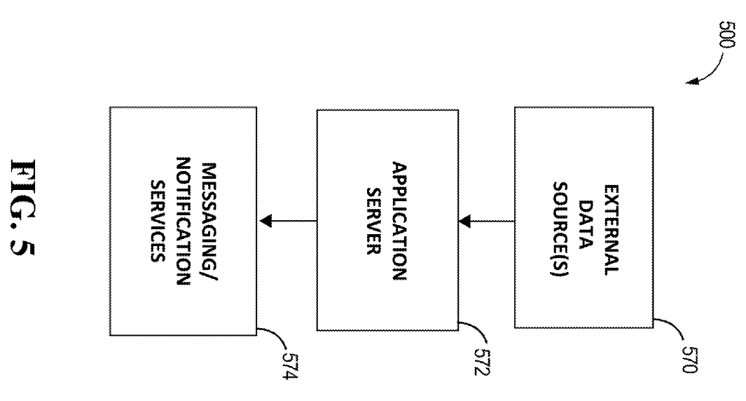

Referring to FIG. 5, a system 500 for providing notifications and messaging in connection with behavioral habit formation is illustrated. The system 500 may comprise a computer-implemented system for behavioral habit formation with privacy-preserving social accountability. The system 500 may include external data sources 570, an application server 572, and messaging/notification services 574. The system 500 may enable retrieval of external data, processing of the external data according to privacy-preserving rules, and delivery of appropriate notifications while excluding sensitive underlying data from communications sent to circle participants.

With continued reference to FIG. 5, the external data sources 570 may be positioned at a top portion of the system 500 and may provide data to the application server 572. The external data sources 570 may include third-party financial data providers or other sources of user-linked resource data that may be used to determine goal-related information. In some examples, the external data sources 570 may enable real-time account linking with dynamic balance retrieval, allowing the system 500 to access current financial information associated with user accounts. In some implementations, the external data sources 570 may provide data that enables goal visualization to be dynamically linked to one or more external bank accounts with real-time or near-real-time balance retrieval. The external data sources 570 may communicate with the application server 572 using vendor-agnostic functional integration patterns that enable data retrieval from various external sources without dependency on specific vendors or geographic regions.

The application server 572 may receive data from the external data sources 570 and may process the data to support goal management, progress tracking, and social accountability functions. In some examples, the application server 572 may utilize an event-driven backend architecture for processing system operations. Further, in some examples, the event-driven backend architecture may enable the application server 572 to respond to events such as goal creation, progress updates, visibility designation changes, and user interactions in an asynchronous manner. In some examples, the application server 572 may include rules engines that govern behavior, motivation, and visibility logic as separate functional components. Further, in some examples, the rules engines may implement rule-based governance that determines what information is shown, to whom the information is shown, and when the information is displayed. The system architecture of the application server 572 may be designed with logic separation to enable scalability across the distributed system. The logic separation may allow the behavior rules, motivation rules, and visibility rules to be maintained and updated independently while operating together to support the overall functionality of the system 500.

As further shown in FIG. 5, the messaging/notification services 574 may be connected to the application server 572 and may be positioned at a bottom portion of the system 500. The messaging/notification services 574 may receive instructions from the application server 572 and may be configured to transmit various communications to user devices. The messaging/notification services 574 may transmit abstracted progress signals to invited participants in a user's circle. The abstracted progress signals may comprise milestone indicators, percentage values, or streak indicators that convey goal progress without revealing sensitive underlying data such as account balances or transaction histories.

The messaging/notification services 574 may also transmit behavioral nudges in response to detected user inactivity or goal stagnation. The behavioral nudges may be generated by the application server 572 based on monitoring of goal activity and detection of conditions indicative of inactivity or stagnation. The messaging/notification services 574 may deliver the behavioral nudges through push notifications, in-application messages, or other communication channels supported by the system 500.

In some examples, with continued reference to FIG. 5, the messaging/notification services 574 may deliver daily educational prompts that are tied to user behavior and progress within the system 500. Further, in some examples, the daily educational prompts may be segmented by mastery level, such as beginner, intermediate, or advanced levels, and may be delivered contextually based on user behavior and goal progress. The messaging/notification services 574 may also generate non-monetary social signaling to provide accountability without financial disclosure. The non-monetary social signaling may enable invited participants to receive progress updates and encouragement signals without exposure to sensitive financial information associated with the goal owner.

The flow of data in the system 500 may proceed from the external data sources 570 through the application server 572 to the messaging/notification services 574. The application server 572 may retrieve data from the external data sources 570, process the data according to privacy-preserving rules implemented by the rules engines, and instruct the messaging/notification services 574 to deliver appropriate notifications to user devices. The event-driven backend architecture of the application server 572 may enable responsive processing of data changes and user interactions, while the logic separation may support scalability as the number of users and goals increases within the system 500.

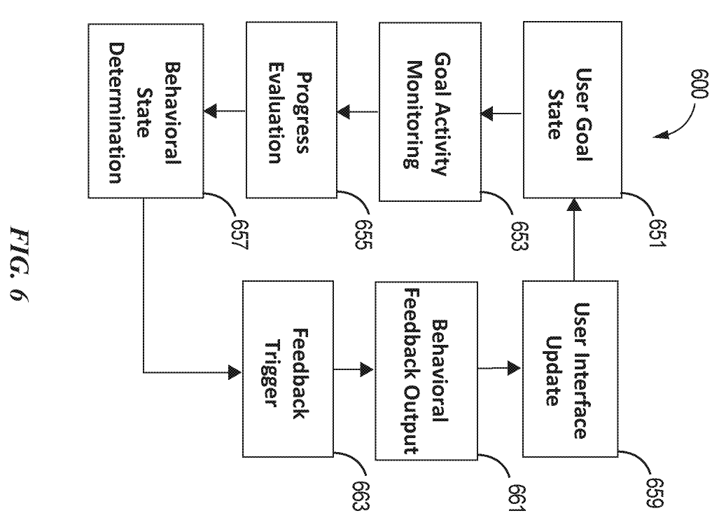

Referring to FIG. 6, a method 600 for behavioral feedback in a habit formation system is illustrated. The method 600 may be performed by a computer-implemented system for behavioral habit formation with privacy-preserving social accountability. In some implementations, the method 600 may be performed by the application server 124 of FIG. 1 or the application server 224 of FIG. 2. The method 600 may provide a structured approach to detecting user progress, determining behavioral states, and delivering appropriate feedback to support habit formation through adaptive reinforcement mechanisms.

The method 600 may begin with a step 651, where a user goal state is established or accessed. The goal management module 232 may establish the user goal state when a user creates a new goal through the UI/API interface 230. In some cases, the goal management module 232 may access an existing user goal state by retrieving goal data from the datastore 228. The user goal state may include information such as goal parameters, current progress, target values, and the current state within the goal lifecycle. The user goal state may serve as a foundation for subsequent monitoring and evaluation operations within the method 600.

With continued reference to FIG. 6, the method 600 may proceed to a step 653, which involves goal activity monitoring to track user actions related to the goal. The notification module 238 may monitor goal activity by tracking user interactions with the system, progress updates, and other goal-related events. The goal activity monitoring at the step 653 may include tracking the frequency of user logins, the timing of manual allocations toward goals, changes in linked account balances, and other actions that indicate user engagement with the goal. The monitored goal activity data may be stored in the datastore 228 for use in subsequent evaluation operations.

Following the goal activity monitoring at the step 653, the method 600 may move to a step 655, where progress evaluation is performed to assess the user's advancement toward the goal. The progress abstraction module 240 may perform the progress evaluation by comparing current goal progress against target values and historical progress data. The progress evaluation at the step 655 may determine whether the user is on track to achieve the goal within any specified timeframe, whether progress has accelerated or decelerated, and whether the user has achieved any milestones since the previous evaluation. The progress evaluation may generate progress metrics that inform subsequent behavioral state determination.

As further shown in FIG. 6, the method 600 may continue to a step 657, where behavioral state determination is conducted to identify the current behavioral status of the user. The behavioral state determination at the step 657 may classify the user's current engagement level based on the monitored goal activity and progress evaluation results. The behavioral state may indicate whether the user is actively engaged with the goal, whether the user has become inactive, or whether the user's progress has stagnated. The notification module 238 may apply predefined rules to determine the behavioral state based on thresholds for activity frequency, progress rate, and other behavioral indicators.

The method 600 may then proceed to a step 663, which involves a feedback trigger that initiates the feedback process based on the determined behavioral state. The feedback trigger at the step 663 may evaluate the behavioral state determined at the step 657 against criteria that indicate when feedback should be provided to the user. The feedback trigger may be activated when the behavioral state indicates user inactivity, goal stagnation, achievement of a milestone, or other conditions that warrant feedback delivery. The notification module 238 may implement the feedback trigger logic to determine the appropriate type and timing of feedback based on the behavioral state.

In response to the feedback trigger at the step 663, the method 600 may move to a step 661, where behavioral feedback output is generated and provided to the user. The notification module 238 may generate the behavioral feedback output based on the behavioral state and the feedback trigger conditions. The behavioral feedback output may include behavioral nudges, motivational messages, progress acknowledgments, or educational content designed to support continued engagement with the goal.

With continued reference to FIG. 6, in some examples, the system may assign each user a Money Mastery Level (MML) based on responses to structured financial knowledge and behavior quizzes. The MML may represent a categorized assessment of the user's financial understanding and habits. The system may administer quizzes through the UI/API interface 230 and may store quiz responses in the datastore 228. Based on the quiz responses, the system may calculate an MML classification for the user that indicates the user's current level of financial knowledge and behavioral proficiency.

In some examples, the notification module 238 may deliver daily financial tips that are segmented by mastery level including beginner, intermediate, and advanced categories. The daily financial tips may be selected based on the user's assigned MML to provide content that is appropriate for the user's current level of financial understanding. Users assigned to a beginner MML category may receive foundational financial education content, while users assigned to intermediate or advanced MML categories may receive more sophisticated financial guidance. The MML-segmented tip delivery may enable the system to provide contextually relevant educational content that supports behavioral habit formation.

The Money Mastery Level may be periodically recalculated as new quiz data is collected from the user. The system may prompt users to complete additional quizzes at defined intervals or in response to behavioral triggers. As users complete additional quizzes, the system may update the MML classification to reflect changes in the user's financial knowledge and behavioral proficiency. The periodic MML recalculation may enable the system to adapt the educational content delivered to users as their understanding and habits evolve over time. While an MML is described above as one example, further examples are not so limited and may include other types of approaches or methods.

As further shown in FIG. 6, the method 600 may then continue to a step 659, where a user interface update is performed to reflect the feedback and any changes in goal status. The UI/API interface 230 may transmit updated information to client computing devices 122 to display the behavioral feedback, updated progress information, and any changes to the user goal state. The user interface update at the step 659 may include displaying awarded digital badges, updated progress visualizations, MML-segmented educational tips, and other feedback elements generated during the method 600.

The user interface update at the step 659 may connect back to the user goal state at the step 651, creating a continuous feedback loop that enables ongoing monitoring and reinforcement of user behavior. The continuous feedback loop may allow the method 600 to repeatedly cycle through goal activity monitoring, progress evaluation, behavioral state determination, feedback triggering, feedback output generation, and user interface updates. The continuous feedback loop may support sustained behavioral habit formation by providing ongoing adaptive feedback based on the user's evolving goal state and behavioral patterns.

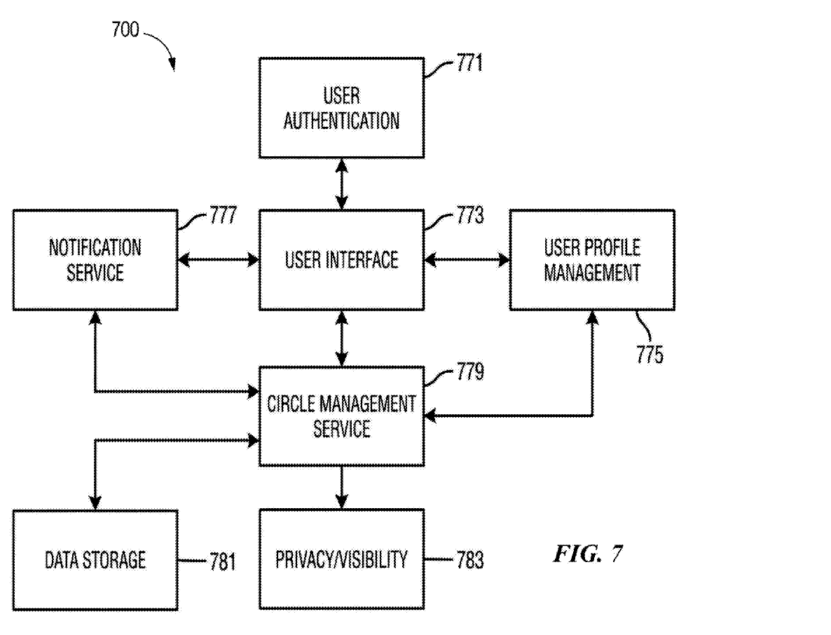

Referring to FIG. 7, a system 700 for managing user circles and privacy-preserving social accountability is illustrated. The system 700 may comprise a computer-implemented system for behavioral habit formation with privacy-preserving social accountability. The system 700 may include a user authentication 771, a user interface 773, a user profile management 775, a notification service 777, a circle management service 779, a data storage 781, and a privacy/visibility 783. The various components of the system 700 may be interconnected to enable management of user-specific circle data structures, processing of visibility designations for goals, and transmission of abstracted progress information to invited participants while preserving privacy of sensitive data.

With continued reference to FIG. 7, the user authentication 771 may be connected to the user interface 773 and may handle verification of user identity and access credentials. The user authentication 771 may receive authentication requests from client computing devices through the user interface 773 and may verify user credentials against stored authentication data. The user authentication 771 may implement authentication mechanisms such as password verification, multi-factor authentication, biometric authentication, or token-based authentication to confirm user identity before granting access to the system 700. Upon successful authentication, the user authentication 771 may establish a session that enables the authenticated user to interact with the various services of the system 700.

The user interface 773 may serve as a primary interaction point and may be connected to the user authentication 771, the user profile management 775, the notification service 777, and the circle management service 779. Through these connections, the user interface 773 may facilitate communication between the user and the various services of the system 700. The user interface 773 may receive requests from client computing devices 122 and may route the requests to appropriate services based on the type of operation requested. The user interface 773 may also receive responses from the services and may transmit the responses to the client computing devices 122 for display to users. In some implementations, the user interface 773 may provide application programming interfaces (APIs) that enable programmatic access to the functionality of the system 700.

As further shown in FIG. 7, the user profile management 775 may be connected to the user interface 773 and the circle management service 779. The user profile management 775 may enable management of user-specific data and preferences. The user profile management 775 may store and retrieve user profile information including user identifiers, contact information, notification preferences, and other user-specific settings. The user profile management 775 may also maintain associations between users and their assigned Money Mastery Levels, digital badges, and other profile attributes. The connection between the user profile management 775 and the circle management service 779 may enable the circle management service 779 to access user profile information when managing circle data structures and processing circle-related operations.

The notification service 777 may be connected to the user interface 773 and the circle management service 779. The notification service 777 may generate and deliver notifications related to circle activities and user interactions. The notification service 777 may transmit abstracted progress signals to invited participants when goals are designated as shared. The notification service 777 may also deliver behavioral nudges in response to detected user inactivity or goal stagnation. The connection between the notification service 777 and the circle management service 779 may enable the notification service 777 to identify invited participants for notification delivery based on circle data structures maintained by the circle management service 779.

With continued reference to FIG. 7, the circle management service 779 may serve as a central component and may be connected to the user interface 773, the user profile management 775, the notification service 777, the data storage 781, and the privacy/visibility 783. The circle management service 779 may manage creation, modification, and maintenance of user-specific circle data structures that identify invited participants for social accountability purposes. The circle management service 779 may maintain a circle data structure associated with a first user, the circle data structure identifying one or more invited participants. The circle data structure may be independent from circle data structures associated with other users, such that each user maintains a separate and non-pooled social graph.

The circle management service 779 may support goal-specific circle assignment capabilities. The circle management service 779 may allow different goals to be associated with different circles, enabling goal-specific social visibility rules. In some implementations, the circle management service 779 may receive a respective visibility designation for each goal of a plurality of goals associated with a user and may enforce goal-level visibility rules such that different goals are shared with different subsets of the one or more invited participants. A user may designate a first goal to be shared with a first subset of invited participants and may designate a second goal to be shared with a second subset of invited participants, and the circle management service 779 may maintain these goal-specific circle assignments within the data storage 781.

As further shown in FIG. 7, the data storage 781 may be connected to the circle management service 779 and may provide persistent storage for circle data, user information, and related records. The data storage 781 may store circle data structures, goal data, visibility designations, user profile information, and other data used by the system 700. The data storage 781 may comprise one or more databases, file systems, or other persistent storage mechanisms. The circle management service 779 may retrieve circle data from the data storage 781 when processing requests related to circle management and may store updated circle data to the data storage 781 as users modify their circle data structures or visibility settings.

The privacy/visibility 783 may be connected to the circle management service 779 and may enforce visibility rules that govern what information is shared with invited participants. The privacy/visibility 783 may ensure that abstracted progress signals are transmitted while sensitive underlying data remains protected. The privacy/visibility 783 may implement rule-based governance that determines what information is shown, to whom the information is shown, and when the information is displayed. The privacy/visibility 783 may evaluate visibility designations stored in the data storage 781 to determine whether goal progress information should be transmitted to invited participants identified in the circle data structure.

With continued reference to FIG. 7, the privacy/visibility 783 may enforce non-custodial fund allocation within the system 700. The privacy/visibility 783 may enforce that allocations update goal progress while preserving full user control over underlying external accounts. The system 700 may generate a total goal representation comprising a first goal portion allocated toward the goal and a second goal portion not allocated toward the goal. The total goal representation may be informational and may not modify underlying user data or access to external resources. The privacy/visibility 783 may ensure that the total goal representation reflects user allocations without modifying actual account ownership, access, or control over external financial accounts linked to the user.

In some implementations, the system 700 may receive allocation data indicating at least one of a manual allocation or an automatic allocation of progress toward the goal. The system 700 may update the first goal portion based on the allocation data without modifying access to the circle data structure. Manual allocations may occur when users explicitly assign portions of funds to specific goals through the user interface 773. Automatic allocations may occur when the system 700 allocates funds to goals based on predefined rules, triggers, or schedules configured by the user. The privacy/visibility 783 may process the allocation data to update the first goal portion within the total goal representation while maintaining the separation between allocation data and circle data structures. The allocation functionality may enable users to mentally partition available resources toward goals without requiring custodial control over those resources and without affecting the visibility settings or circle memberships associated with the goals.

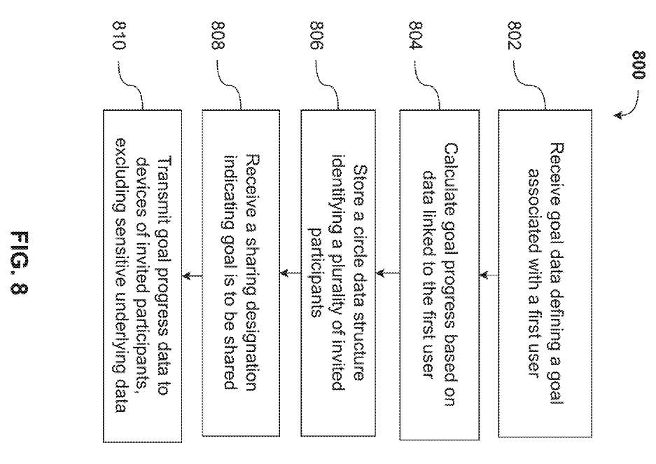

Referring to FIG. 8, a method 800 for providing social accountability in habit formation while preserving privacy is illustrated. The method 800 may be performed by a computer-implemented system for behavioral habit formation with privacy-preserving social accountability. In some implementations, the method 800 may be performed by the application server 124 of FIG. 1 or the application server 224 of FIG. 2. The method 800 may provide a computer-implemented method for providing social accountability in habit formation while preserving privacy by enabling transmission of goal progress information to invited participants while ensuring that sensitive underlying data associated with the goal is not disclosed.

The method 800 may begin with a step 802, where the system receives goal data defining a goal associated with a first user. The goal data may be received by a computing system through the UI/API interface 230 from a client computing device associated with the first user. The goal data may include information such as a goal name, a target amount, a target date, or other parameters that define the goal. The goal management module 232 may process the received goal data and store the goal data in the datastore 228. In some cases, the goal data may define a savings goal, a behavioral objective, or another type of goal that the first user seeks to achieve.

With continued reference to FIG. 8, the method 800 may proceed to a step 804, where the system calculates goal progress based on data linked to the first user. The computing system may calculate goal progress by evaluating data associated with one or more user-linked resources. The progress abstraction module 240 may retrieve data from the datastore 228 or from external service providers 126 to determine current progress toward the goal. The goal progress calculation may incorporate user-provided data indicating manual contributions toward the goal or data retrieved from external data sources indicating changes in linked account balances. The calculated goal progress may comprise abstracted progress output such as a milestone indicator, a percentage value, or a streak indicator.

The method 800 may then proceed to a step 806, where the system stores a circle data structure identifying a plurality of invited participants. The computing system may store a circle data structure associated with the first user in the datastore 228. The circle data structure may identify a plurality of invited participants with whom the first user may share goal progress. The circle management module 236 may maintain the circle data structure and may enable the first user to add or remove invited participants from the circle data structure. The circle data structure may be independent from circle data structures associated with other users, such that each user maintains a separate and non-pooled social graph for social accountability purposes.

As further shown in FIG. 8, the method 800 may continue to a step 808, where the system receives a sharing designation indicating that the goal is to be shared. The computing system may receive a sharing designation indicating that the goal is to be shared with the plurality of invited participants identified in the circle data structure. The privacy/visibility module 234 may receive the sharing designation through the UI/API interface 230 and may store the sharing designation in the datastore 228 in association with the goal. The sharing designation may indicate that the first user has elected to share progress information for the goal with the invited participants.

Following receipt of the sharing designation at the step 808, the method 800 may proceed to a step 810, where the system transmits goal progress data to devices of invited participants, excluding sensitive underlying data. The computing system may transmit the goal progress data to devices associated with the plurality of invited participants. The transmitting may exclude transmission of sensitive underlying data associated with the goal. The notification module 238 may transmit the goal progress data through the network 120 to client computing devices 122 associated with the invited participants. The transmitted goal progress data may comprise abstracted progress information such as milestone indicators, percentage values, or streak indicators, while excluding sensitive data such as account balances, transaction histories, or other financial information.

With continued reference to FIG. 8, the method 800 may support both manual and automatic allocation modes for tracking progress toward goals. The goal management module 232 may support both manual allocations where users explicitly assign funds to goals and automatic allocations based on predefined rules, triggers, or schedules. In manual allocation mode, the first user may explicitly assign portions of funds to the goal through the UI/API interface 230. In automatic allocation mode, the system may allocate funds to the goal based on predefined rules configured by the first user, triggers that activate upon specified conditions, or schedules that define periodic allocation intervals.

The computing system may generate a total goal representation comprising a first goal portion allocated toward the goal and a second goal portion not allocated toward the goal. The total goal representation may be informational and may not modify access to the circle data structure. The progress abstraction module 240 may maintain the total goal representation by tracking the first goal portion and the second goal portion based on allocation data received from the first user or generated by automatic allocation rules. The total goal representation may enable the first user to visualize progress toward the goal without modifying underlying user data or access to external resources. The allocation functionality may update goal progress while preserving full user control over underlying external accounts and without affecting the circle data structure or visibility settings associated with the goal.

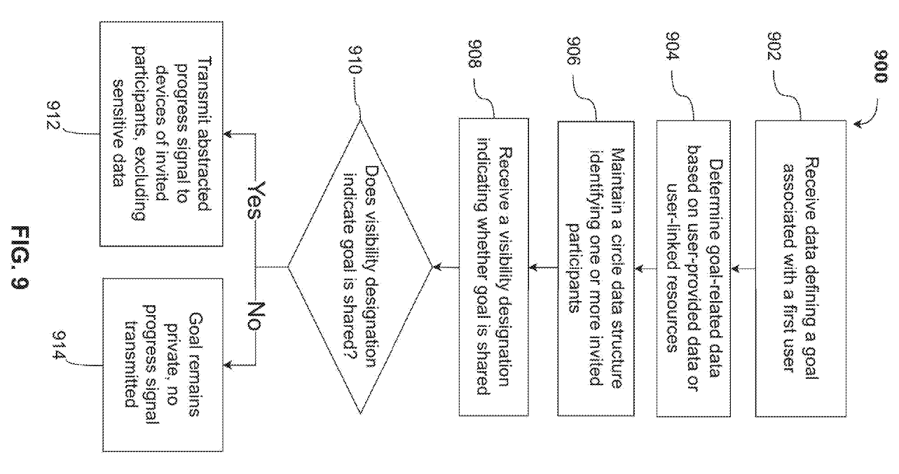

Referring to FIG. 9, a method 900 for behavioral habit formation with privacy-preserving social accountability is illustrated. The method 900 may be performed by a computer-implemented system for behavioral habit formation with privacy-preserving social accountability. In some implementations, the method 900 may be performed by the application server 124 of FIG. 1 or the application server 224 of FIG. 2. The method 900 may provide a computer-implemented method for behavioral habit formation with privacy-preserving social accountability by enforcing visibility designations that govern whether goal progress information is shared with invited participants and by transmitting abstracted progress signals that exclude sensitive underlying data when sharing is enabled.

The method 900 may begin with a step 902, where the system receives data defining a goal associated with a first user. The data defining the goal may be received by a computing system through the UI/API interface 230 from a client computing device associated with the first user. The data defining the goal may include information such as a goal name, a target amount, a target date, or other parameters that characterize the goal. The goal management module 232 may process the received data and store the data in the datastore 228. In some cases, the data defining the goal may specify a savings objective, a behavioral target, or another type of goal that the first user seeks to achieve.

With continued reference to FIG. 9, the method 900 may proceed to a step 904, where the system determines goal-related data based on user-provided data or data associated with user-linked resources. The computing system may determine goal-related data by evaluating at least one of user-provided data or data associated with one or more user-linked resources. The progress abstraction module 240 may retrieve data from the datastore 228 or from the external service providers 126 to determine current progress toward the goal. User-provided data may include manual entries indicating contributions toward the goal, while data associated with user-linked resources may include balance information retrieved from external financial accounts linked to the first user. The goal-related data may be used to calculate progress toward the goal and to generate abstracted progress information for potential transmission to invited participants.

The method 900 may then proceed to a step 906, where the system maintains a circle data structure identifying one or more invited participants. The computing system may maintain a circle data structure associated with the first user. The circle data structure may identify one or more invited participants with whom the first user may share goal progress. The circle management module 236 may maintain the circle data structure in the datastore 228 and may enable the first user to add or remove invited participants from the circle data structure. The circle data structure may be independent from circle data structures associated with other users, such that each user maintains a separate and non-pooled social graph for social accountability.

As further shown in FIG. 9, the method 900 may continue to a step 908, where the system receives a visibility designation indicating whether the goal is shared. The computing system may receive a visibility designation through the UI/API interface 230 from a client computing device associated with the first user. The privacy/visibility module 234 may receive the visibility designation and may store the visibility designation in the datastore 228 in association with the goal. The visibility designation may indicate whether the first user has elected to share progress information for the goal with the one or more invited participants identified in the circle data structure.

Following receipt of the visibility designation at the step 908, the method 900 may proceed to a step 910, which involves a decision point to determine whether the visibility designation indicates the goal is shared. The privacy/visibility module 234 may evaluate the visibility designation to determine whether the goal is designated as shared with the invited participants. The decision point at the step 910 may branch the method 900 into two paths based on the visibility designation.

With continued reference to FIG. 9, if the visibility designation indicates the goal is shared (Yes branch from the step 910), the method 900 may proceed to a step 912, where the system transmits an abstracted progress signal to devices associated with the invited participants, excluding sensitive data. The computing system may transmit the abstracted progress signal to devices associated with the one or more invited participants identified in the circle data structure. The notification module 238 may transmit the abstracted progress signal through the network 120 to client computing devices 122 associated with the invited participants. The abstracted progress signal may comprise abstracted progress information such as a milestone indicator, a percentage toward goal completion, or a streak indicator. The abstracted progress signal may exclude sensitive data such as account balances, transaction histories, or other financial information associated with the goal.

If the visibility designation does not indicate the goal is shared (No branch from the step 910), the method 900 may proceed to a step 914, where the goal remains private and no progress signal is transmitted. The step 914 may be reached when the visibility designation indicates that the goal is not shared with invited participants. When the goal remains private, no progress information is transmitted to external participants, and the goal data remains accessible to the goal owner through the client computing devices 122. The privacy/visibility module 234 may enforce the private designation by preventing transmission of any progress information to invited participants for the goal.

As further shown in FIG. 9, the circle data structure maintained at the step 906 may be independent from circle data structures associated with other users, such that each user maintains a separate and non-pooled social graph. The circle data structure may comprise unidirectional visibility, wherein a first user may expose progress for one or more goals to members of a selected circle without receiving access to goals of those members unless such members independently designate goals as shared with the first user. The circle data structure may comprise viewer-specific views, wherein different invited participants may receive different views of goal progress based on goal-level visibility rules configured by the first user. The circle data structure may comprise asymmetric disclosure, wherein the first user may share goal progress with invited participants without creating reciprocal obligations for the invited participants to share their own goal progress with the first user.

In some implementations, the method 900 may further comprise detecting, by the computing system, at least one of user inactivity or goal stagnation based on the goal progress. The notification module 238 may monitor goal activity by tracking user interactions with the system, progress updates, and other goal-related events stored in the datastore 228. User inactivity may be detected when the first user has not interacted with the system or updated goal progress within a specified time period. Goal stagnation may be detected when goal progress has remained unchanged or has decreased over a defined interval despite the goal remaining in an active state.

With continued reference to FIG. 9, in response to detecting the at least one of user inactivity or goal stagnation, the method 900 may further comprise generating and transmitting, by the computing system, a behavioral nudge to a device associated with the first user. The notification module 238 may generate the behavioral nudge based on the detected inactivity or stagnation conditions. The behavioral nudge may comprise motivational messages, reminders about goal deadlines, suggestions for actions to advance toward the goal, or other content designed to re-engage the first user with the goal. The notification module 238 may transmit the behavioral nudge through the network 120 to a client computing device associated with the first user.

The method 900 may further comprise awarding, by the computing system, a digital badge to the first user based on a defined behavior. The defined behavior may comprise at least one of consistency in activity, maintenance of a goal streak, or goal completion. The notification module 238 may evaluate user behavior against defined criteria to determine when a digital badge should be awarded. The digital badges may function as behavioral reinforcement mechanisms that recognize and reward positive behaviors associated with goal progress. The awarded digital badges may be stored in the datastore 228 in association with a user profile of the first user and may be displayed through the client computing devices 122 to provide ongoing motivation for continued engagement with goals.

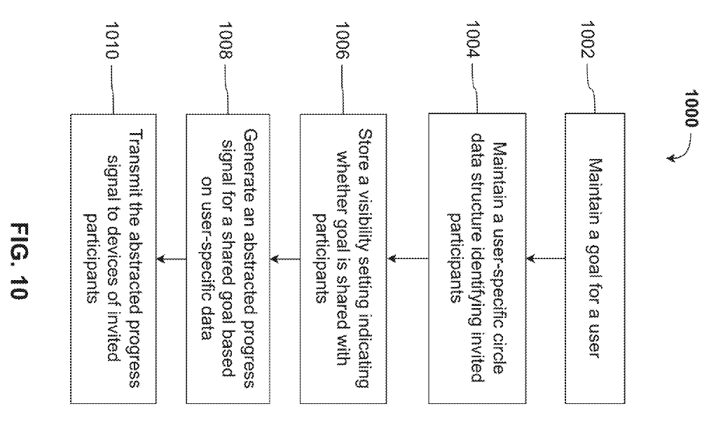

Referring to FIG. 10, a method 1000 for providing social accountability in habit formation while preserving privacy is illustrated. The method 1000 may be performed by a computer-implemented system for behavioral habit formation with privacy-preserving social accountability. In some implementations, the method 1000 may be performed by the application server 124 of FIG. 1 or the application server 224 of FIG. 2. The method 1000 may be embodied as instructions stored on a non-transitory computer-readable medium that, when executed by one or more processors, cause the one or more processors to perform operations for providing social accountability in habit formation while preserving privacy. The non-transitory computer-readable medium may comprise any suitable storage medium including magnetic storage media, optical storage media, solid-state storage media, or other non-volatile storage media capable of storing instructions executable by a processor.

The method 1000 may begin with a step 1002, where the system maintains a goal for a user. The goal management module 232 may maintain the goal by storing goal data in the datastore 228 and tracking the current state of the goal within a goal lifecycle. Maintaining the goal may include storing goal parameters such as a goal name, a target amount, a target date, or other attributes that define the goal. The goal management module 232 may also maintain the goal by tracking progress toward the goal based on user-provided data or data associated with one or more user-linked resources. In some cases, the goal may be associated with a goal state selected from a group comprising created, active, paused, completed, and abandoned, and the goal management module 232 may update the goal state as the user interacts with the goal over time.

With continued reference to FIG. 10, the method 1000 may proceed to a step 1004, where the system maintains a user-specific circle data structure identifying invited participants. The circle management module 236 may maintain the user-specific circle data structure in the datastore 228. The user-specific circle data structure may identify one or more invited participants with whom the user may share goal progress. The circle data structure may be independent from circle data structures associated with other users, such that each user maintains a separate and non-pooled social graph. The independence of the circle data structure may enable each user to manage a distinct set of invited participants without affecting or being affected by circle data structures maintained by other users within the system environment 100. The circle management module 236 may enable the user to add or remove invited participants from the user-specific circle data structure through the UI/API interface 230.

The method 1000 may then proceed to a step 1006, where the system stores a visibility setting indicating whether the goal is shared with participants identified in the user-specific circle data structure. The privacy/visibility module 234 may store the visibility setting in the datastore 228 in association with the goal. The visibility setting may indicate whether the user has elected to share progress information for the goal with the invited participants identified in the user-specific circle data structure. In some implementations, the visibility setting may be configured at the goal level, allowing the user to designate some goals as private and other goals as shared with particular invited participants. The privacy/visibility module 234 may enforce the visibility setting when determining whether to transmit progress information to invited participants.

As further shown in FIG. 10, the method 1000 may continue to a step 1008, where the system generates an abstracted progress signal for a shared goal based on user-specific data. The progress abstraction module 240 may generate the abstracted progress signal by applying a data abstraction layer that transforms user-specific data into non-sensitive progress information suitable for sharing with invited participants. The abstracted progress signal may comprise at least one of a milestone indicator, a percentage toward goal completion, or a streak indicator. The milestone indicator may indicate that the user has achieved a defined milestone in progress toward the goal. The percentage toward goal completion may indicate the proportion of the goal that has been achieved relative to a target value. The streak indicator may indicate a consecutive period during which the user has maintained consistent activity toward the goal. The abstracted progress signal may exclude sensitive data such as account balances, transaction histories, or other financial information associated with the goal.

Following the generation of the abstracted progress signal at the step 1008, the method 1000 may proceed to a step 1010, where the system transmits the abstracted progress signal to devices associated with the invited participants. The notification module 238 may transmit the abstracted progress signal through the network 120 to client computing devices 122 associated with the invited participants identified in the user-specific circle data structure. The transmission may deliver the abstracted progress information to the invited participants while excluding sensitive underlying data from the communication. In this manner, the method 1000 may enable social accountability through visibility of progress rather than through disclosure of sensitive underlying data. The invited participants may receive the abstracted progress signal and may view the progress information through client applications executing on the client computing devices 122.

With continued reference to FIG. 10, the user-specific circle data structure maintained at the step 1004 may support unidirectional visibility, asymmetric disclosure, or viewer-specific views. The unidirectional visibility may enable a first user to expose progress for one or more goals to members of a selected circle without receiving access to goals of those members unless such members independently designate goals as shared with the first user. The asymmetric disclosure may allow the first user to share goal progress with invited participants without creating reciprocal obligations for the invited participants to share their own goal progress with the first user. The viewer-specific views may enable different invited participants to receive different views of goal progress based on goal-level visibility rules configured by the first user. The separate and non-pooled social graph maintained for each user may enable privacy-preserving social accountability without commingling user data or creating shared data structures that span multiple users.

In some implementations, the operations performed by the method 1000 may further comprise detecting at least one of user inactivity or goal stagnation. The notification module 238 may monitor goal activity by tracking user interactions with the system, progress updates, and other goal-related events stored in the datastore 228. User inactivity may be detected when the user has not interacted with the system or updated goal progress within a specified time period. Goal stagnation may be detected when goal progress has remained unchanged or has decreased over a defined interval despite the goal remaining in an active state. The notification module 238 may evaluate the monitored goal activity data against predefined thresholds or patterns to identify conditions indicative of inactivity or stagnation.

In response to detecting the at least one of user inactivity or goal stagnation, the operations may further comprise generating and transmitting a behavioral nudge to a device associated with the user. The notification module 238 may generate the behavioral nudge based on the detected inactivity or stagnation conditions. The behavioral nudge may comprise motivational messages, reminders about goal deadlines, suggestions for actions to advance toward the goal, or other content designed to re-engage the user with the goal. The notification module 238 may transmit the behavioral nudge through the network 120 to a client computing device associated with the user. The behavioral nudge may function as a reinforcement mechanism that encourages the user to resume progress toward the goal when activity has stalled. The detection of inactivity or stagnation and the generation of behavioral nudges may operate in conjunction with the social accountability features of the method 1000 to support behavioral habit formation through adaptive reinforcement mechanisms.

The methods described herein, including the method 300, the method 400, the method 600, the method 800, the method 900, and the method 1000, may be performed by the systems described herein. In some implementations, the system environment 100 of FIG. 1 may perform one or more of the methods described herein. The application server 124 may execute instructions stored in memory to perform the operations of the methods, including receiving goal data, maintaining circle data structures, processing visibility designations, generating abstracted progress signals, and transmitting progress information to invited participants. The application server 224 of FIG. 2 may similarly perform the methods through the coordinated operation of the UI/API interface 230, the goal management module 232, the privacy/visibility module 234, the circle management module 236, the notification module 238, and the progress abstraction module 240. The system 500 of FIG. 5 may perform the methods by retrieving data from the external data sources 570, processing the data through the application server 572, and delivering notifications through the messaging/notification services 574. The system 700 of FIG. 7 may perform the methods through the coordinated operation of the user authentication 771, the user interface 773, the user profile management 775, the notification service 777, the circle management service 779, the data storage 781, and the privacy/visibility 783. In some cases, the methods may be performed by a combination of components from multiple systems described herein. The systems may be configured to perform the methods in various sequences, and certain operations of the methods may be performed in parallel, omitted, or supplemented with additional operations depending on the particular implementation.

A number of implementations have been described. Nevertheless, it will be understood that various modifications may be made without departing from the spirit and scope of the disclosure. Accordingly, other implementations are within the scope of the following claims.

The systems and methods described herein may be implemented in various configurations and combinations. The embodiments and examples presented throughout this disclosure are illustrative and are not intended to limit the scope of the disclosed subject matter. Different features described in connection with separate embodiments may be combined in ways not explicitly described, and features described in connection with a single embodiment may be implemented separately or in different combinations with other features. The particular arrangements of components, modules, and processing steps depicted in the figures and described in the specification represent example implementations, and other arrangements may be employed without departing from the principles described herein. The order of operations in the methods described herein may be varied in some implementations, and certain operations may be performed in parallel, omitted, or supplemented with additional operations. The specific data structures, communication protocols, and system architectures described herein may be modified or substituted with alternative approaches that achieve similar functionality. References to particular hardware components, software modules, or network configurations are provided as examples and do not preclude the use of other technologies or implementations that provide equivalent capabilities.

### EXAMPLES

In accordance with the systems and methods described herein, a computer-implemented system for behavioral habit formation with privacy-preserving social accountability may comprise a processor and a memory coupled to the processor and storing instructions that, when executed by the processor, cause the system to receive data defining a goal associated with a first user. The instructions may further cause the system to determine goal-related data based on at least one of user-provided data or data associated with one or more user-linked resources. The instructions may further cause the system to maintain a circle data structure associated with the first user, the circle data structure identifying one or more invited participants. The instructions may further cause the system to receive a visibility designation indicating whether the goal is shared with the one or more invited participants. In response to the visibility designation indicating the goal is shared, the instructions may cause the system to transmit to devices associated with the one or more invited participants an abstracted progress signal derived from the goal, wherein the progress signal comprises abstracted progress information and excludes sensitive data associated with the goals.

In some implementations, the progress signal may comprise at least one of a milestone indicator, a percentage toward goal completion, or a streak indicator. The instructions may further cause the system to generate the progress signal by applying a data abstraction layer that transforms the progress data into abstracted progress information. The circle data structure may be independent from circle data structures associated with other users, such that each user maintains a separate and non-pooled social graph.

In some aspects, the instructions may further cause the system to receive data defining a plurality of goals associated with the first user, receive a respective visibility designation for each goal of the plurality of goals, and enforce goal-level visibility rules such that different goals are shared with different subsets of the one or more invited participants. Each goal of the plurality of goals may be associated with a goal state selected from a group comprising created, active, paused, completed, and abandoned.

In some cases, the instructions may further cause the system to detect at least one of user inactivity or goal stagnation and, in response to detecting the at least one of user inactivity or goal stagnation, generate and transmit a behavioral nudge to a user device associated with the first user. The instructions may further cause the system to award a digital badge to the first user based on a defined behavior, the defined behavior comprising at least one of consistency in savings activity, maintenance of a streak, or goal completion.

In some implementations, the instructions may further cause the system to generate a total goal representation comprising a first goal portion allocated toward the goal and a second goal portion not allocated toward the goal, wherein the total goal representation is informational and does not modify underlying user data or access to external resources. The instructions may further cause the system to receive allocation data indicating at least one of a manual allocation or an automatic allocation of progress toward the goal and update the first goal portion based on the allocation data without modifying access to the circle data structure.

In accordance with the methods described herein, a computer-implemented method for providing social accountability in habit formation while preserving privacy may comprise receiving, by a computing system, goal data defining a goal associated with a first user. The method may further comprise calculating, by the computing system, goal progress based on data linked to the first user. The method may further comprise storing, by the computing system, a circle data structure associated with the first user, the circle data structure identifying a plurality of invited participants. The method may further comprise receiving, by the computing system, a sharing designation indicating that the goal is to be shared with the plurality of invited participants. The method may further comprise transmitting, by the computing system, the goal progress data to devices associated with the plurality of invited participants, wherein the transmitting excludes transmission of sensitive underlying data associated with the goal.

In some aspects, the circle data structure may be independent from circle data structures associated with other users, such that each user maintains a separate and non-pooled social graph for social accountability, the circle data structure comprises unidirectional visibility, the circle data structure comprises viewer-specific views, or the circle data structure comprises asymmetric disclosure. The goal progress may comprise abstracted progress output and the abstracted progress output may comprise at least one of a milestone indicator, a percentage value, or a streak indicator.

In some cases, the method may further comprise generating, by the computing system, a total goal representation comprising a first goal portion allocated toward the goal and a second goal portion not allocated toward the goal, wherein the total goal representation is informational and does not modify access to the circle data structure. The method may further comprise detecting, by the computing system, at least one of user inactivity or goal stagnation based on the goal progress and, in response to detecting the at least one of user inactivity or goal stagnation, generating and transmitting, by the computing system, a behavioral nudge to a device associated with the first user. The method may further comprise awarding, by the computing system, a digital badge to the first user based on a defined behavior, the defined behavior comprising at least one of consistency in activity, maintenance of a goal streak, or goal completion.

In accordance with the non-transitory computer-readable media described herein, a non-transitory computer-readable medium storing instructions that, when executed by one or more processors, cause the one or more processors to perform operations may be provided. The operations may comprise maintaining a goal for a user. The operations may further comprise maintaining a user-specific circle data structure identifying invited participants. The operations may further comprise storing a visibility setting indicating whether the goal is shared with participants identified in the user-specific circle data structure. The operations may further comprise generating, for a shared goal, an abstracted progress signal based on user-specific data. The operations may further comprise transmitting the abstracted progress signal to devices associated with the invited participants.

In some implementations, the abstracted progress signal may comprise at least one of a milestone indicator, a percentage toward goal completion, or a streak indicator. The circle data structure may be independent from circle data structures associated with other users, such that each user maintains a separate and non-pooled social graph. The operations may further comprise detecting at least one of user inactivity or goal stagnation and, in response to detecting the at least one of user inactivity or goal stagnation, generating and transmitting a behavioral nudge to a device associated with the user.

1. A computer-implemented system for behavioral habit formation with privacy-preserving social accountability, comprising:
a processor; and
a memory coupled to the processor and storing instructions that, when executed by the processor, cause the system to:
receive data defining a goal associated with a first user;
determine goal-related data based on at least one of user-provided data or data associated with one or more user-linked resources;
maintain a circle data structure associated with the first user, the circle data structure identifying one or more invited participants;
receive a visibility designation indicating whether the goal is shared with the one or more invited participants; and
in response to the visibility designation indicating the goal is shared, transmit to devices associated with the one or more invited participants an abstracted progress signal derived from the goal, wherein the progress signal comprises abstracted progress information and excludes sensitive data associated with the goals.

2. The system of claim 1, wherein the progress signal comprises at least one of a milestone indicator, a percentage toward goal completion, or a streak indicator.

3. The system of claim 2, wherein the instructions further cause the system to generate the progress signal by applying a data abstraction layer that transforms the progress data into abstracted progress information.

4. The system of claim 1, wherein the circle data structure is independent from circle data structures associated with other users, such that each user maintains a separate and non-pooled social graph.

5. The system of claim 1, wherein the instructions further cause the system to:
receive data defining a plurality of goals associated with the first user;
receive a respective visibility designation for each goal of the plurality of goals; and
enforce goal-level visibility rules such that different goals are shared with different subsets of the one or more invited participants.

6. The system of claim 5, wherein each goal of the plurality of goals is associated with a goal state selected from a group comprising created, active, paused, completed, and abandoned.

7. The system of claim 1, wherein the instructions further cause the system to:
detect at least one of user inactivity or goal stagnation; and
in response to detecting the at least one of user inactivity or goal stagnation, generate and transmit a behavioral nudge to a user device associated with the first user.

8. The system of claim 7, wherein the instructions further cause the system to award a digital badge to the first user based on a defined behavior, the defined behavior comprising at least one of consistency in savings activity, maintenance of a streak, or goal completion.

9. The system of claim 1, wherein the instructions further cause the system to:
generate a total goal representation comprising a first goal portion allocated toward the goal and a second goal portion not allocated toward the goal, wherein the total goal representation is informational and does not modify underlying user data or access to external resources.

10. The system of claim 9, wherein the instructions further cause the system to:
receive allocation data indicating at least one of a manual allocation or an automatic allocation of progress toward the goal; and
update the first goal portion based on the allocation data without modifying access to the circle data structure.

11. A computer-implemented method for providing social accountability in habit formation while preserving privacy, the method comprising:
receiving, by a computing system, goal data defining a goal associated with a first user;
calculating, by the computing system, goal progress based on data linked to the first user;
storing, by the computing system, a circle data structure associated with the first user, the circle data structure identifying a plurality of invited participants;
receiving, by the computing system, a sharing designation indicating that the goal is to be shared with the plurality of invited participants; and
transmitting, by the computing system, the goal progress data to devices associated with the plurality of invited participants, wherein the transmitting excludes transmission of sensitive underlying data associated with the goal.

12. The method of claim 11, wherein the circle data structure is independent from circle data structures associated with other users, such that:
each user maintains a separate and non-pooled social graph for social accountability;
the circle data structure comprises unidirectional visibility;
the circle data structure comprises viewer-specific views; or
the circle data structure comprises asymmetric disclosure.

13. The method of claim 11, wherein the goal progress comprises abstracted progress output and the abstracted progress output comprises at least one of a milestone indicator, a percentage value, or a streak indicator.

14. The method of claim 11, further comprising:
generating, by the computing system, a total goal representation comprising a first goal portion allocated toward the goal and a second goal portion not allocated toward the goal, wherein the total goal representation is informational and does not modify access to the circle data structure.

15. The method of claim 11, further comprising:
detecting, by the computing system, at least one of user inactivity or goal stagnation based on the goal progress; and
in response to detecting the at least one of user inactivity or goal stagnation, generating and transmitting, by the computing system, a behavioral nudge to a device associated with the first user.

16. The method of claim 15, further comprising:
awarding, by the computing system, a digital badge to the first user based on a defined behavior, the defined behavior comprising at least one of consistency in activity, maintenance of a goal streak, or goal completion.

17. A non-transitory computer-readable medium storing instructions that, when executed by one or more processors, cause the one or more processors to perform operations comprising:
maintaining a goal for a user;
maintaining a user-specific circle data structure identifying invited participants;
storing a visibility setting indicating whether the goal is shared with participants identified in the user-specific circle data structure;
generating, for a shared goal, an abstracted progress signal based on user-specific data; and
transmitting the abstracted progress signal to devices associated with the invited participants.

18. The non-transitory computer-readable medium of claim 17, wherein the abstracted progress signal comprises at least one of a milestone indicator, a percentage toward goal completion, or a streak indicator.

19. The non-transitory computer-readable medium of claim 17, wherein the circle data structure is independent from circle data structures associated with other users, such that each user maintains a separate and non-pooled social graph.

20. The non-transitory computer-readable medium of claim 17, wherein the operations further comprise:
detecting at least one of user inactivity or goal stagnation; and
in response to detecting the at least one of user inactivity or goal stagnation, generating and transmitting a behavioral nudge to a device associated with the user.
